(19) United States
(12) Patent Application Publication
(10) Pub. No.: US 2005/0049289 A1
(43) Pub. Date: Mar. 3, 2005

(54) Pramipexole for the treatment ADHD

(75) Inventors: Juergen Reess, Ulm (DE); Franco Borsini, Bad Waldsee (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/958,156

(22) Filed: Oct. 4, 2004

Related U.S. Application Data
(63) Continuation of application No. 10/198,480, filed on Jul. 18, 2002, now abandoned.
(60) Provisional application No. 60/312,241, filed on Aug. 14, 2001.

(30) Foreign Application Priority Data
Aug. 3, 2001 (DE) 101 37 633.2

(51) Int. Cl. A61K 31/428

(52) U.S. Cl. 514/367

(57) ABSTRACT

A method for prevention and/or treatment of ADHD in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof

### RELATED APPLICATIONS

This application is a continuation of U.S. Ser. No. 10/198,480 filed Jul. 18, 2002, which claims benefit under 35 U.S.C. § 119(e) of prior provisional application Ser. No. 60/312,241, filed Aug. 14, 2001.

### FIELD OF THE INVENTION

The invention relates to the use of 2-amino-4,5,6,7-tetrahydro-6-n-propylaminobenzothiazole, the (+) or (−) enantiomer thereof, the pharmacologically acceptable acid addition salts thereof, as well as hydrates and solvates, for preparing a pharmaceutical composition for the prevention and/or treatment of ADHD.

### BACKGROUND OF THE INVENTION

2-Amino-6-n-propylamino-4,5,6,7-tetrahydrobenzothiazole is a dopamine agonist which is also known in the art by the name pramipexole. Pramipexole and processes for preparing it are known for example from EP-A-186 087 and U.S. Pat. No. 4,886,812. It is known in particular for the treatment of schizophrenia and particularly for the treatment of Parkinson's disease. The prior art further discloses, for example, that pramipexole lowers the prolactin serum level (DE 38 43 227).

### DESCRIPTION OF THE INVENTION

Surprisingly, it has been found that pramipexole can be used in therapeutically effective doses for the prevention and/or treatment of ADHD.

Accordingly, the present invention relates to the use of 2-amino-4,5,6,7-tetrahydro-6-n-propylaminobenzothiazole, optionally in the form of its (+) or (−) enantiomer, optionally in the form of the pharmacologically acceptable acid addition salts, as well as hydrates and solvates, for preparing a pharmaceutical composition for the prevention and/or treatment of ADHD.

The abbreviation ADHD stands for “attention deficit hyperactivity disorder”. It denotes a disorder which affects both children and adults in the form of an attention deficit. ADHD also refers to a hyperactivity disorder which is also observed both in children and adults. Depending on which of the above disorders predominates, ADHD may be observed in different degrees. There is the combined type in which both an attention deficit and a hyperactivity disorder can be observed, the predominantly inattentive type and the predominantly hyperactive-impulsive type.

The combined type is present when at least six out of nine symptoms of the attention disorder and hyperactivity/impulsivity persist for at least six months. If at least six symptoms of attention deficit but fewer than six of hyperactivity/impulsivity have persisted for at least six months it is the predominantly inattentive type. The predominantly hyperactive-impulsive type is present when at least six symptoms of hyperactivity/impulsivity but fewer than six of attention deficit have persisted for a period of at least six months.

The following criteria of assessment are symptoms of inattentiveness and hyperactivity, which form the basis for classifying the abovementioned types of ADHD (according to the Diagnostic and Statistical Manual of Psychic Disorders, Version IV (DSM IV):

Inattentiveness
frequently takes no notice of details or makes errors of haste in schoolwork, at work or in other activities;
often has difficulty maintaining concentration for lengthy periods during tasks or when playing;
frequently appears not to be listening when spoken to by other people;
often does not filly carry out instructions from other people and cannot complete schoolwork, other work or duties in the workplace;
frequently has problems organizing tasks and activities;
frequently avoids, is disinclined to do or often only undertakes under protest tasks which require sustained mental effort;
frequently loses objects that he/she needs for tasks or activities;
is often easily distracted by external stimuli;
is often forgetful during everyday activities;

Hyperactivity
frequently fidgets with hands or feet or shifts around on the chair;
often stands up in class or in other situations where people are expected to stay seated;
frequently runs around or climbs up excessively in situations where this is inappropriate (in young people or adults this may be restricted to a subjective feeling of restlessness);
often has trouble playing quietly or occupying him/herself quietly with leisure activities; is often “on the go” or frequently acts as if he/she were “driven”;
often talks excessively; impulsiveness;
often bursts out with the answers before the question is finished;
has trouble waiting his/her turn;
often interrupts and disturbs other people (e.g., interrupts conversations or breaks into other people's games).

Accordingly, the present invention relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of attention deficits.

Moreover, the present invention relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of hyperactivity disorders.

In another aspect the present invention relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of hyperactivity disorders accompanied by attention deficit.

For treatment and/or prevention of the medical indications described above, in addition to monotherapy using pramipexole it is also possible, as an alternative, to carry out a combined therapy using pramipexole with one or more, preferably one other pharmaceutically active compound. It may prove effective, for example, to use a combination with active substances selected from among the α-sympathomimetics and antidepressants, preferably tricyclic antidepressants, antidepressants of the SSRI, or antidepressants of the MAO type.

In the case of a combined therapy, pramipexole may preferably be used to treat the abovementioned conditions with one or more, preferably one of the following substances: methylphenidate, amphetamine, amphetaminil, methamphetamine, pemoline, tomoxetin, desipramine, imipramine, bupropion, and modafmil, of which methylphenidate, amphetamine, pemoline, tomoxetin, and modafinil are particularly preferred.

Pramipexole may be used within the scope of the present invention as a racemate or in the form of its (+) or (−) enantiomer. Moreover, pramipexole may be used in the form of the pharmaceutically acceptable acid addition salts thereof as well as optionally in the form of its hydrates and/or solvates. By pharmaceutically acceptable acid addition salts are meant according to the invention the salts selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid, of which the salts of hydrochloric acid, hydrobromic acid, sulfuric acid, phosphoric acid, and acetic acid are particularly preferred. The salts of hydrochloric acid are particularly important. Most preferably, within the scope of the present invention, therefore, the hydrochlorides of pramipexole are used, pramipexole dihydrochloride being of particular significance. Of the hydrates of pramipexole, pramipexole dihydrochloride monohydrate is particularly preferred.

The dosage of pramipexole naturally depends to a great extent on the clinical picture. For example, without restricting the present invention thereto, pramipexole may be used in doses of about 0.05 mg to 7.5 mg, preferably 0.1 mg to 5 mg per day. These doses are based on pramipexole in the form of its free base. Based on the salt form pramipexole dihydrochloride monohydrate which is preferably used, the doses mentioned above correspond to about 0.07 mg to 10.65 mg, preferably 0.14 mg to 7.1 mg of pramipexole dihydrochloride monohydrate per day.

One possible dosing method, which is to be understood as being merely an illustrative example, is described below, based on pramipexole in the form of its free base: individual dosage titration at weekly intervals depending on activity and acceptability.
1st week: 1 tablet containing 0.088 mg of pramipexole 3 times a day;
2nd week: 1 tablet containing 0.18 mg of pramipexole 3 times a day; and
3rd week and thereafter: ½ tablet containing 0.7 mg of pramipexole 3 times a day.

Within the scope of the use according to the invention pramipexole may be administered orally, transdermally, intrathecally, by inhalation or parenterally. Suitable preparations include for example tablets, capsules, suppositories, solutions, syrups, emulsions, dispersible powders, or patches. Regarding possible embodiments of a transdermal preparation which may be used according to the invention, we now refer to the embodiments described by way of example in U.S. Pat. No. 5,112,842, which is hereby incorporated by reference in its entirety. Suitable tablets may be produced, for example, by mixing the active substance or substances with known excipients, for example, inert diluents, such as calcium carbonate, calcium phosphate, or lactose, disintegrants such as corn starch or alginic acid, binders such as starch or gelatine, lubricants such as magnesium stearate or talc, and/or agents for achieving delayed release such as carboxymethylcellulose, cellulose acetate phthalate, or polyvinyl acetate. The tablets may also consist of several layers.

The following are some examples of pharmaceutical preparations which may be used according to the invention. These are intended solely as an illustration without restricting the subject matter of the invention thereto.

Tablet 1

| Ingredient | Amount (mg) |
|---|---|
| pramipexole dihydrochloride monohydrate | 1.00 |
| mannitol | 121.50 |
| maize starch | 79.85 |
| highly dispersed silicon dioxide, anhydrous | 2.30 |
| Polyvidone K25 | 2.35 |
| magnesium stearate | 3.00 |
| Total | 210.00 |

Tablet 2

| Ingredient | Amount (mg) |
|---|---|
| pramipexole | 0.5 |
| mannitol | 122.0 |
| maize starch, dried | 61.8 |
| maize starch | 18.0 |
| highly dispersed silicon dioxide, anhydrous | 2.4 |
| Polyvidone K25 | 2.3 |
| magnesium stearate | 3.0 |
| Total | 210.0 |

Tablet 3

| Ingredient | Amount (mg) |
|---|---|
| pramipexole | 0.25 |
| mannitol | 61.0 |
| maize starch | 39.90 |
| highly dispersed silicon dioxide, anhydrous | 1.20 |
| Polyvidone K25 | 1.15 |
| magnesium stearate | 1.5 |
| Total | 105.00 |

Tablet 4

| Ingredient | Amount (mg) |
|---|---|
| pramipexole | 0.125 |
| mannitol | 49.455 |
| maize starch, dried | 25.010 |
| maize starch | 7.300 |
| highly dispersed silicon dioxide, anhydrous | 0.940 |
| Polyvidone K25 | 0.940 |
| magnesium stearate | 1.230 |
| Total | 85.000 |

Formulation Suitable for Injection

| Ingredient | Amount |
|---|---|
| pramipexole dihydrochloride monohydrate | 0.3 mg |
| sodium chloride | 0.8 mg |
| benzalkonium chloride | 0.01 mg |
| water for injection | ad 100 mL |

1. A method for prevention and/or treatment of ADHD in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

2. The method of claim 1, wherein the 2-amino-4,5,6,7-tetrahydro-6-n-propylaminobenzothiazole is the (+) or (−) enantiomer thereof.

3. A method for prevention and/or treatment of attention deficits in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

4. The method of claim 3, wherein the 2-amino-4,5,6,7-tetrahydro-6-n-propylaminobenzothiazole is the (+) or (−) enantiomer thereof.

5. A method for prevention and/or treatment of hyperactivity disorders in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

6. The method of claim 5, wherein the 2-amino-4,5,6,7-tetrahydro-6-n-propylaminobenzothiazole is the (+) or (−) enantiomer thereof.

7. A method for prevention and/or treatment of hyperactivity disorders accompanied by attention deficits in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

8. The method of claim 7, wherein the 2-amino-4,5,6,7-tetrahydro-6-n-propylaminobenzothiazole is the (+) or (−) enantiomer thereof.

9. A method for prevention and/or treatment of ADHD in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

10. The method of claim 9, wherein the 2-amino-4,5,6,7-tetrahydro-6-n-propylaminobenzothiazole is the (+) or (−) enantiomer thereof.

11. The method of claim 10, wherein the second pharmaceutical substance is an α-sympathomimetic or antidepressant.

12. The method of claim 10, wherein the second pharmaceutical substance is selected from methylphenidate, amphetamine, amphetaminil, methamphetamine, pemoline, tomoxetin, desipramine, imipramine, bupropion, and modafinil.

13. A method for prevention and/or treatment of attention deficits in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

14. The method of claim 13, wherein the 2-amino-4,5,6,7-tetrahydro-6-n-propylaminobenzothiazole is the (+) or (−) enantiomer thereof.

15. The method of claim 14, wherein the second pharmaceutical substance is an α-sympathomimetic or antidepressant.

16. The method of claim 14, wherein the second pharmaceutical substance is selected from methylphenidate, amphetamine, amphetaminil, methamphetamine, pemoline, tomoxetin, desipramine, imipramine, bupropion, and modafinil.

17. A method for prevention and/or treatment of hyperactivity disorders in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

18. The method of claim 17, wherein the 2-amino-4,5,6,7-tetrahydro-6-n-propylaminobenzothiazole is the (+) or (−) enantiomer thereof.

19. The method of claim 17, wherein the second pharmaceutical substance is an α-sympathomimetic or antidepressant.

20. The method of claim 17, wherein the second pharmaceutical substance is selected from methylphenidate, amphetamine, amphetaminil, methamphetamine, pemoline, tomoxetin, desipramine, imipramine, bupropion, and modafinil.

21. A method for prevention and/or treatment of hyperactivity disorders accompanied by attention deficits in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

22. The method of claim 21, wherein the 2-amino-4,5,6,7-tetrahydro-6-n-propylaminobenzothiazole is the (+) or (−) enantiomer thereof.

23. The method of claim 22, wherein the second pharmaceutical substance is an α-sympathomimetic or antidepressant.

24. The method of claim 22, wherein the second pharmaceutical substance is selected from methylphenidate, amphetamine, amphetaminil, methamphetamine, pemoline, tomoxetin, desipramine, imipramine, bupropion, and modafinil.

25. The method according to claim 1, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

26. The method according to claim 1, wherein the 2-amino-4,5,6,7-tetrahydro-6-n-propylaminobenzothiazole is used in a dose of about 0.05 mg to 7.5 mg per day.

27. The method according to claim 1, wherein the 2-amino-4,5,6,7-tetrahydro-6-n-propylaminobenzothiazole is used in a dose of about 0.1 mg to 5.0 mg per day.

28. The method according to claim 3, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

29. The method according to claim 3, wherein the 2-amino-4,5,6,7-tetrahydro-6-n-propylaminobenzothiazole is used in a dose of about 0.05 mg to 7.5 mg per day.

30. The method according to claim 3, wherein the 2-amino-4,5,6,7-tetrahydro-6-n-propylaminobenzothiazole is used in a dose of about 0.1 mg to 5.0 mg per day.

31. The method according to claim 5, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

32. The method according to claim 5, wherein the 2-amino-4,5,6,7-tetrahydro-6-n-propylaminobenzothiazole is used in a dose of about 0.05 mg to 7.5 mg per day.

33. The method according to claim 5, wherein the 2-amino-4,5,6,7-tetrahydro-6-n-propylaminobenzothiazole is used in a dose of about 0.1 mg to 5.0 mg per day.

34. The method according to claim 7, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

35. The method according to claim 7, wherein the 2-amino-4,5,6,7-tetrahydro-6-n-propylaminobenzothiazole is used in a dose of about 0.05 mg to 7.5 mg per day.

36. The method according to claim 7, wherein the 2-amino-4,5,6,7-tetrahydro-6-n-propylaminobenzothiazole is used in a dose of about 0.1 mg to 5.0 mg per day.
